(19) United States
(12) Patent Application Publication
(10) Pub. No.: US 2011/0137391 A1
(43) Pub. Date: Jun. 9, 2011

(54) FOLDABLE COIL FOR AN IMPLANTABLE MEDICAL DEVICE

(76) Inventor: C. Roger Leigh, New South Wales (AU)

(21) Appl. No.: 12/996,332

(22) PCT Filed: Jun. 3, 2009
(86) PCT No.: PCT/AU09/00696

§ 371 Date: Feb. 16, 2011

(30) Foreign Application Priority Data
Jun. 3, 2008 (AU) 2008902796

(51) Int. Cl. A61N 1/00

(52) U.S. Cl. 607/116

(57) ABSTRACT

An implantable component of a medical device including an implantable coil including a conductor disposed in a carrier including first and second fold lines, wherein the carrier has at least one structural variation along each of the fold lines such that a substantially straight edge is formed along each of the fold lines when the coil is biased into a folded configuration, and an electronics module electrically connected to the coil and configured to inductively communicate, via the coil, with an external component of the device.

### CROSS-REFERENCE TO RELATED APPLICATIONS

This application is a National Stage Application of International Patent Application No. PCT/AU2009/000696, filed Jun. 3, 2009, which claims the benefit of Australian Provisional Application No. 2008902796, filed Jun. 3, 2008, both of which are hereby incorporated by reference herein.

### BACKGROUND

1. Field of the Invention

Aspects and embodiments of the present invention relate to structures for implantable devices, and particularly to a foldable coil for an implantable medical device.

2. Related Art

An implantable device, such as a medical device or any other device adapted to be implanted within a user's body, may include an implantable structure. Some implantable structures are required to have a substantial operative area, dimension or volume. Examples of such structures are antennas and coils for use in transcutaneous inductive power and/or data transfer between the implanted device and an external device. Such arrangements are used, for example, in cochlear prosthesis systems and other hearing prostheses.

Cochlear prostheses typically include an external component having an external coil and an implanted component having an implanted coil to form a transcutaneous link of the medical implant. The coils are arranged to provide an inductive coupling so as to facilitate the transfer of data and power through the skin of the patient.

### SUMMARY

In accordance with one aspect of the present invention, an implantable component of a medical device is provided. The implantable component comprises an implantable coil comprising a conductor disposed in a carrier including first and second fold lines, wherein the carrier has at least one structural variation along each of the fold lines such that a substantially straight edge is formed along each of the fold lines when the coil is biased into a folded configuration, and an electronics module electrically connected to the coil and configured to inductively communicate, via the coil, with an external component of the device.

In accordance with another aspect of the present invention, a system is provided. The system comprises an implantable component of a medical device including an implantable coil comprising a conductor disposed in a carrier including first and second fold lines, wherein the carrier has at least one structural variation along each of the fold lines such that a substantially straight edge is formed along each of the fold lines when the coil is biased into a folded configuration, and an electronics module electrically connected to the coil and configured to inductively communicate, via the coil, with an external component of the device. The system further comprises an insertion tool configured to maintain the coil the folded configuration when the coil is operatively engaged with the insertion tool, wherein the coil assumes a substantially planar unfolded configuration when the coil is removed from operative engagement with the insertion tool.

In accordance with another aspect of the present invention, a method of implanting a component of a medical device is provided. The component of the medical device comprises an implantable coil including a conductor disposed in a carrier including first and second fold lines and an electronics module electrically connected to the coil and configured to inductively communicate, via the coil, with an external component of the device. The method comprises folding the coil, along each of the fold lines, into a folded configuration, wherein the carrier has at least one structural variation along each of the fold lines such that a substantially straight edge is formed along each of the fold lines when the coil is folded, inserting the coil into an incision in a user's body along an axis substantially parallel to each of the fold lines, and allowing the coil to assume an unfolded configuration in the user's body, wherein the width of the coil substantially perpendicular to the fold lines in the unfolded configuration is greater than the width of the coil substantially perpendicular to the fold lines in the folded configuration.

### BRIEF DESCRIPTION OF THE DRAWINGS

Illustrative embodiments of the present invention will now be described with reference to the accompanying figures, in which:

FIG. 1 is a cross-sectional view illustrating a prior art implant;

FIG. 2 is a schematic view of the implant of FIG. 1;

FIG. 3 is a plan view of the implant of FIG. 1;

FIG. 4A is an illustration of one embodiment of the present invention in an open position;

FIG. 4B is an illustration of the embodiment of FIG. 4A in a folded position;

FIG. 5A is an illustration of an embodiment of the present invention in an open position;

FIG. 5B is an illustration of the embodiment of FIG. 5A in a folded position;

FIG. 6A is an illustration of a third implementation of the present invention in an open position;

FIG. 6B is an illustration of the third implementation of FIG. 6A in a folded position;

FIGS. 7A and 7B illustrate the process of surgical insertion of the implementation of FIG. 6B;

FIGS. 8A-8E illustrate modifications to the carrier to facilitate folding in certain implementations of the present invention;

FIG. 8F-8J illustrate modifications to the coil in certain implementations of the present invention; and

FIGS. 9A and 9B illustrate another alternative implementation of the present invention.

### DETAILED DESCRIPTION

Aspects and embodiments of the present invention will be described with reference to a particular illustrative example, which is a coil device intended for use in a cochlear implant system. However, it will be appreciated that embodiments of the present invention are applicable wherever an implanted structure is operatively required to have a relatively large dimension, area or volume. Examples of such structures include reservoirs for drugs or other substances, coil devices for other active implantable medical devices, sensors or stimulation systems, arrays for visual prostheses, or plates and structures used in reconstructive surgery. Embodiments of the present invention encompass both such a structure as an original part of an implanted device, or such a structure being used as a replacement, repair or enhancement to a medical device or system.

Embodiments of the present invention may be applied to any medical device or other device for implantation which includes such an implanted structure. Certain embodiments have particular application to active implanted medical devices, and are particularly applicable where a resonant structure is required, such as a coil or antenna for power and/or data transfer. Such a resonant structure may be implemented as a component of a cochlear implant system, a hybrid electrical/acoustic system, a hearing aid system, or any other suitable hearing prosthesis or other active implantable medical device. It will be appreciated that the exemplary implementations described below are for illustrative purposes, and its features are not intended to be limitative of the scope of the present invention. Many variations and additions are possible within the scope of the present invention.

Embodiments of the present invention are concerned with implantable devices, particularly medical devices but including any device adapted to be implanted within a recipient's body. More particularly, embodiments of the present invention relate to implantable structures, particularly those which are required to have a substantial operative area, dimension or volume.

For the purposes of this specification and claims, “insertion profile” refers to the width and/or shape of the device or relevant structure as presented for insertion through an incision or other opening during implantation.

The implantable structure may be a resonant structure, or may be a different structure. In particular, embodiments of the present invention are applicable to any structure which may limit the use of minimally invasive surgery by reason of a required operative area, dimension or volume. Some specific examples are a neural sensor array, a reservoir for drugs or other substances, or a sensor or stimulation array for a visual prosthesis. The structure may include conductive components. The implantable medical device may include other components which are not adapted to change configuration. Embodiments of the present invention may also be applied to different components of a multi-component implant system. Embodiments of the present invention have particular application to an active implanted medical device.

For the purposes of this specification and claims, the term “conductive structure” is intended to encompass electrically conductive structures, which may for example be resonant structures operatively forming part of a wireless communications or power structure. The term specifically includes coils, of any suitable shape or geometry, which operatively form part of an inductive system. The term also includes antennas, intended to transmit, receive or both, for an RF transmission system.

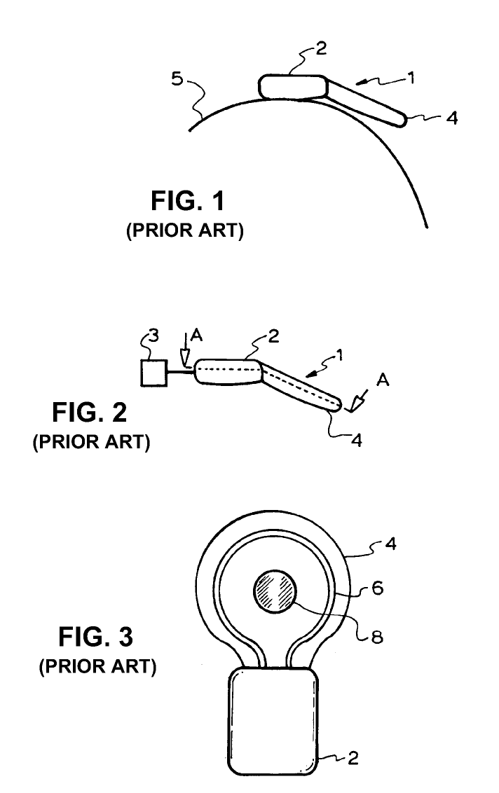

FIGS. 1-3 illustrate a prior art implant. The external component (not shown) includes the transmitter coil, a microphone, and a signal processor to receive, process and inductively transmit audio signals to the implanted component. The implanted component 1 typically includes a receiver coil 6, an implant stimulator 2, and an implant transducer 3 to inductively receive audio data and power, process the audio data, and deliver stimuli. The implanted component 1 of the cochlear prosthesis is usually implanted in or near the mastoid region of the skull 5 behind the ear of the patient, and the transducer element 3 is implanted within the scala tympani so as to provide electrical stimuli to a user. The external component is typically detachably secured on the user's head, so that the external coil is retained in the correct position relative to the implanted coil. This is typically achieved using a magnet disposed in the implanted component to retain a magnet in the external component in the correct position.

Various disclosures address aspects of the inductive link. For example, U.S. Pat. No. 6,327,504 describes the general principles of a common transcutaneous link. U.S. Pat. No. 6,430,444 discloses another transcutaneous energy transfer arrangement that uses multiple coils to control the energy transfer in the transcutaneous link.

Whilst existing coils perform their task satisfactorily, there is a trend for surgical techniques in general to move toward using smaller incisions. Smaller incisions reduce recovery time for the patient and reduce the risk of infection. Current cochlear implant coils are typically over 30 mm in diameter. Surgeons would like a much smaller coil to facilitate a minimally invasive incision technique. However, in order to maximize the efficiency of power transfer, the electrical efficiency of the receiver coil needs to be maximized, and this in turn is dependant upon its area.

In embodiments of the present invention, the implant coil is made such that immediately prior to implantation it is in a folded state, so as to present a smaller insertion profile. Following implantation the coil is allowed to expand to its full width.

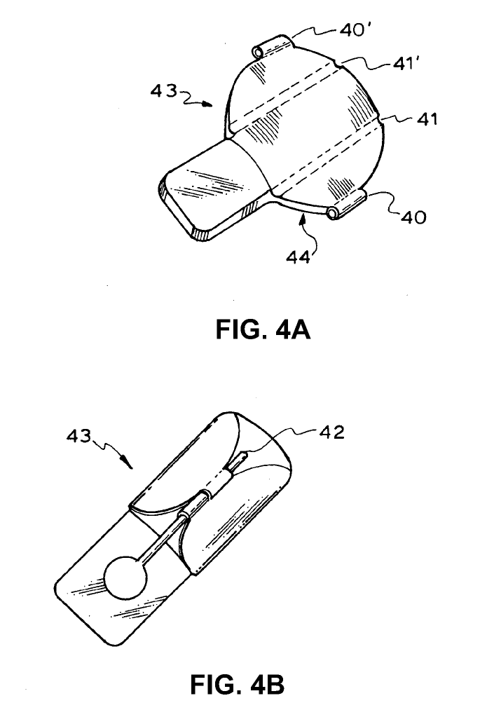

Retaining the coil in the folded state can be achieved in a number of ways. In one embodiment, shown in FIGS. 4A and 4B, the coil structure has lumens or anchor points allowing a stylet to be inserted. FIG. 4A shows this embodiment in the pre-folded state. Lumens 40 and 40′ are provided in the implantable coil 43 to accommodate a stylet 42. FIG. 4B shows implantable coil 43 in its folded state with stylet 42 in place through the lumens 40, 40′, thereby retaining implantable coil 43 in a folded state.

Preferably, implantable coil 43 has fold features 41, 41′ to facilitate folding. Implantable coil 43 includes a conductor (not shown, typically formed from platinum wire) and a carrier 44, typically formed from a moulded elastomer such as silicone. Fold features 41, 41′ may include changes to the conductor and/or changes to carrier 44. In this embodiment, the fold features are provided by a change in cross sectional shape of the conductor such that it is relatively thin in the plane perpendicular to the fold. This modification can be achieved by compressing the wire in a press. It is preferred that the carrier 44 is reduced in thickness at the same locations as the cross sectional change in the conductor.

Implantable coil 43 may either be supplied in the folded state, with stylet 42 inserted, or it may be supplied unfolded and the stylet inserted immediately prior to surgery. The latter option avoids any risk that long term storage in the folded position causes memory effects such that the implantable coil 43 does not easily take up the flat shape following removal of stylet 42.

The folded implantable coil 43 may easily be inserted through a relatively small incision. Once inserted, stylet 42 (or in alternative embodiments, the coil is otherwise expanded) is removed and implantable coil 43 resumes its flat position.

FIGS. 7A and 7B illustrate conceptually a preferred insertion procedure. The opening prepared must be internally of sufficient dimensions to accommodate the implantable coil 60 in its extended position. FIG. 7A shows the folded implantable coil 60, and the incision 70 through skin and appropriate tissue. It can be seen that opening 71 is of sufficient extent to accommodate the implantable coil 60 in its opened position. FIG. 7B shows the implantable coil 60 in its operative, opened position within opening 71. It will be apparent that there must be sufficient room and/or tissue elasticity to allow the coil to transition from the folded to the flat position within the body. The force to cause the transition is provided, in this implementation, by the memory of the conductor and carrier (elastomer) which were originally formed in the flat position and tend to return to that position. Additional mechanisms could be provided to increase this force if required, for example in particular applications, however the inherent force has been generally found to be sufficient.

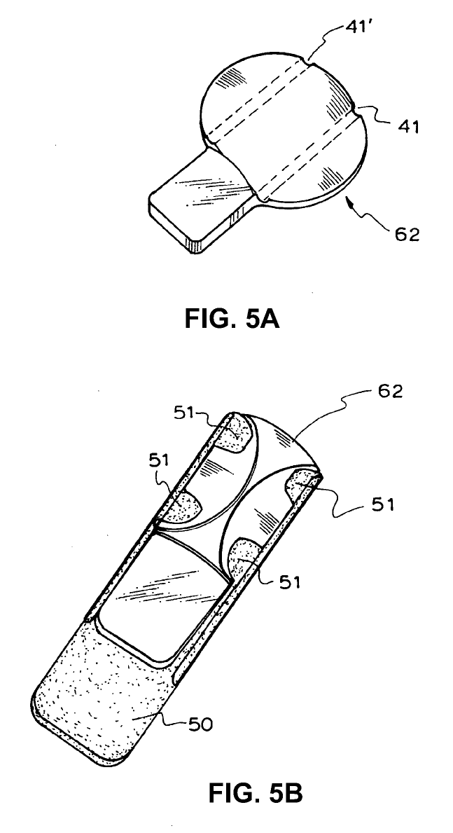

In one alternative embodiment, an insertion tool may be used to hold the implantable coil folded for insertion. After insertion through the skin incision the insertion tool is withdrawn and the coil expands to its full dimensions. The embodiment of FIG. 5A shows the implantable coil 62 in the open position, noting that it is a similar to the embodiment of FIG. 4A, but without lumens. FIG. 5B shows the implantable coil 62 folded within the insertion tool 50, the latter including flanges 51 to hold the coil in the folded position during insertion. After insertion, the insertion tool is withdrawn, and the implantable coil expanded to its unfolded, operative configuration.

In the simplest embodiment, the tool to retain the coil in a folded position could be the surgeon's hand. In this instance, the features to facilitate bending or folding of the coil structure become particularly important, as they must be robust enough to handle folding and compression applied manually (and hence with some variability of force) during surgery.

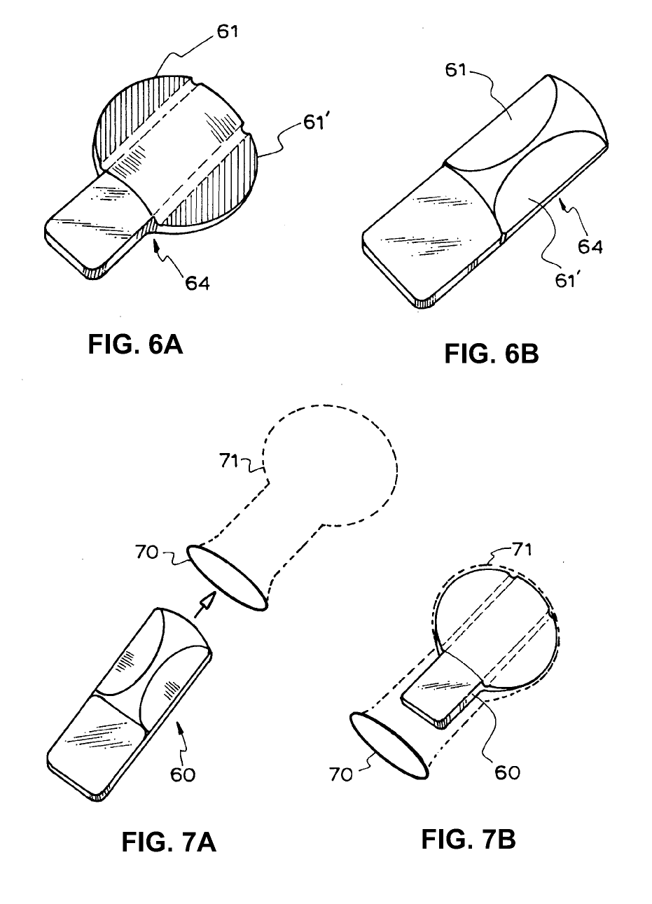

An alternate embodiment uses an adhesive or similar material which dissolves in the body to retain the coil in the folded state. Any suitable biocompatible material could be used, for example polyvinyl acetate (PVA). The adhesive holds the coil in the folded state until implantation and dissolves after implantation allowing the coil to expand to full size. The expansion could be relatively rapid, or slow if the structure is not required to reach full size immediately. In the flat position the implantable coil 64 could be as shown in FIG. 6A. Areas 61 and 61′ would have adhesive applied. The folded position is shown in FIG. 6B. It will be appreciated that other similar effects, for example a temperature or moisture responsive material, could be used to retain the coil in position until after insertion. Similar materials could be used to move the coil from the folded position to open position after insertion into the body.

Another advantage of embodiments of the present invention relates to implant fixation. In order to prevent implants of certain types from moving following implantation they must be restrained in some way. This is particularly the case for implanted coils intended for power transfer, and the AIMDs associated with those coils. It is critical that the coil is correctly oriented to enable efficient power transfer. This restraint may be achieved in a number of ways, for example using sutures; recessing into bone; or using screws and mesh, either individually or in combination.

It is also possible to restrain the implant using available body tissue. For example, in a cochlear implant surgery, the implant is often placed under the periosteum. This very tough tissue is often sufficient to retain the implant. Embodiments of the present invention combined with minimally invasive surgery can enhance this retention. Referring to FIG. 7A, in a preferred implementation, opening 71 is a recess formed under the periosteum, by lifting up the periosteum through incision 70. The folded implantable coil 60 is then inserted through the incision as shown in FIG. 7B. Implantable coil 60 expands into opening 71 so as to assume its operative position. As the periosteum returns to its normal position, the implantable coil is firmly anchored in position by the periosteum.

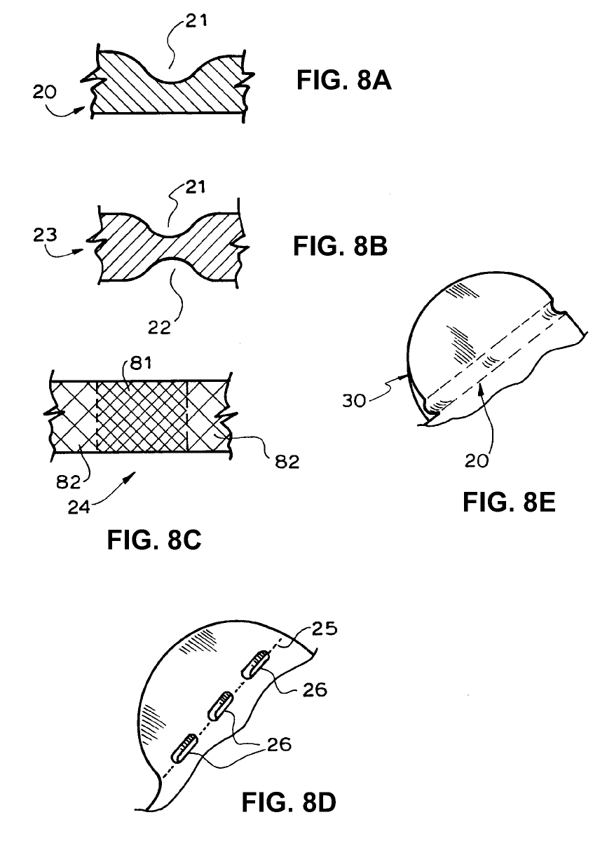

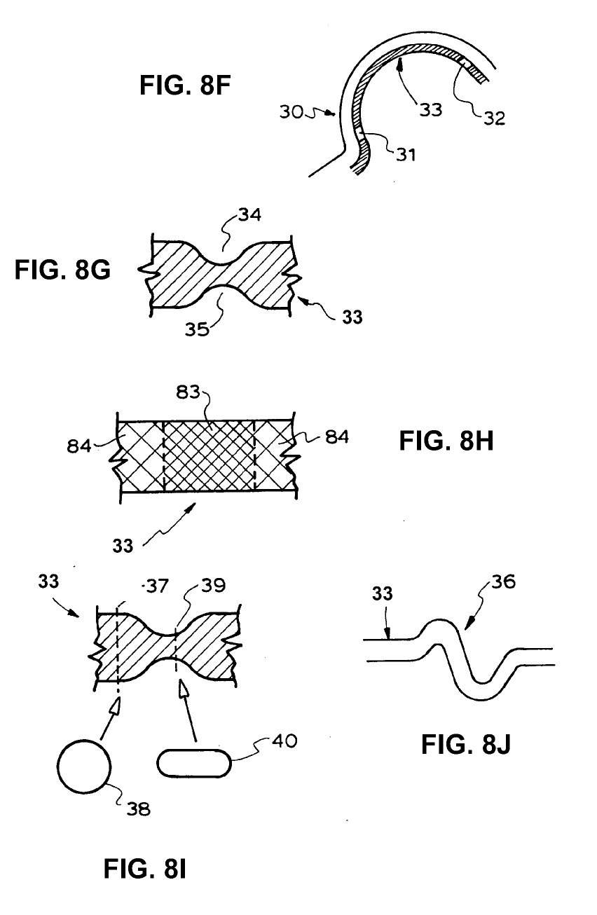

It will be appreciated that there are many ways to define the fold lines according to the illustrated implementations, and that embodiments of the present invention are not restricted to any particular arrangement. For example, the carrier may be modified by a use of a lower durometer silicone in the fold region, or by holes or recesses passing through the thickness of the carrier. FIGS. 8A-8E illustrate several alternative approaches. As shown in FIGS. 8A and 8E, a fold line 20 of carrier 30 may be formed by a channel 21 on one surface so as to form a narrow, natural fold line. In an alternative implementation of a fold line 23, illustrated in FIG. 8B, fold line 23 may be formed from channels 21, 22 on both surfaces.

As illustrated in FIG. 8C, another alternative is to use a lower durometer (stiffness) material 81 along a fold line 24, and a higher durometer material 82 elsewhere. This could be through the entire material, or for only a portion of the thickness. In another alternative, illustrated in FIG. 8D, a fold line 25 is defined by recesses or holes 26.

The coil conductor may be modified, for example, by a reduction in cross sectional area or a reduction in stiffness at the appropriate fold points. This may be achieved by localised heat treatment, or a strain relief mechanism such as a concertinaed section or change in the cross sectional shape. It will be appreciated that these approaches may be used in combination, and that many other alternative approaches may be used to facilitate folding of the conductors.

FIGS. 8F-8J illustrate examples of such techniques. In general, the fold lines in coil carrier 30 are preferably matched by appropriate modifications to the underlying coil conductors 33 at points 31, 32, as shown in FIG. 8F. It will be appreciated that depending upon the structure of the coil, there may be more or fewer points or conductors requiring modification.

One approach is to reduce the cross sectional area, as illustrated in FIG. 8G, by crimping or other processes, as shown by indentations 34, 35. Alternatively, as illustrated in FIG. 8H, the region of the conductor 33 requiring to be bent to facilitate folding may be composed of relatively soft material 83 relative to the less flexible material 84 of the remaining conductor. This may be achieved by appropriate heat treatment, for example.

Another alternative is to provide a mechanical strain relief structure. One approach is to provide a concertina or similar structure 36 within the conductor, as illustrated in FIG. 8J. Another approach is to have a change in cross section. As illustrated in FIG. 8I, the cross-sectional shape of the conductor 33 changes from circular 38 at line 37, to oblate 40 at line 39.

Many of the above methods may also be used in combination. For example, the conductor could be of reduced cross section and reduced hardness in the fold region. Of course, the conductor and coil are preferably modified in the same region to achieve the desired folding.

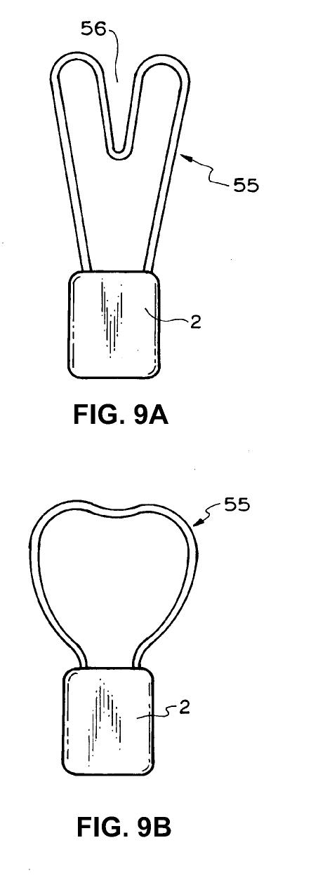

It will be appreciated that there are many additional mechanisms which could be used to reduce the cross section for insertion, for example providing only one fold, providing multiple folds, rolling or bending. The folding mechanism could be also be within the plane of the coil, as shown in FIGS. 9A and 9B. Referring to FIGS. 9A and 9B, coil 55 folds inwardly within the plane of the coil, about fold 56. This may be achieved either by manual force, or by a mechanism, for example such as has been previously discussed for out of plane folding. It can be seen that this allows the coil to have a reduced insertion profile, to facilitate minimally invasive surgery, and then expand to occupy a suitable body opening.

For some applications, it may be desired to provide an active force to assist in fully opening or configuring the structure after insertion. For example, a shape memory alloy or polymer could be used to effect the alteration in shape, with an appropriately selected transition temperature. A resilient member could be retained in position and released after insertion. Materials which otherwise respond to introduction within the body (e.g., to moisture or temperature) could also be used.

Whilst embodiments of the present invention have been predominantly described with reference to a circular wire coil, certain embodiments of the invention could be implemented with differently shaped coils, or with coils formed from a foil or similar material. For example, embodiments of the present invention could be used with some of the non-round coils described in PCT/AU2007/001561. The foil coil described in that disclosure may also be effective to facilitate bending of the coil.

It is noted that embodiments of the present invention can be applied to a coil and carrier as a separate device, or as part of an implant assembly, as desired. It is also envisaged that different arrangements than those described, for example rolling up, may be used to achieve the reduced cross-sectional area for insertion.

The certain embodiments described above relate to folding operations. It will be appreciated that embodiments of the present invention are not limited to these specific embodiments, and encompass other mechanisms and approaches which achieve the same result. For example, the radius of curvature of the coil could be reduced for implantation and increased for operation. There could be two or more relatively small turns during implantation which expand to one relatively large turn operatively. Alternatively part of the coil could be a helix or other structure which is relatively short for insertion and relatively long operatively. The expansion of this helix section could be caused by different relative temperatures external and internal to the body and could be effected by a suitable shape memory alloy.

It will also be understood that embodiments of the present invention intended for other implantable structures will need to be considered in the context of the function of those devices and structures.

In embodiments of the present invention, an implantable medical device is provided, the implantable medical device including a structure which has a first configuration and a second configuration, the first configuration presenting a smaller insertion profile than the second configuration, wherein after the device is implanted, it is adapted to be placed into the second configuration.

In embodiments of the present invention, an implantable medical device is provided, the implantable medical device including a conductive structure which has a first configuration and a second configuration, the first configuration presenting a smaller insertion profile than the second configuration, wherein after the device is implanted, it is adapted to be placed into the second, operative configuration.

In certain embodiments of the present invention, the conductive structure includes a coil adapted to provide an inductive link with another device.

In certain embodiments of the present invention, the conductive structure includes an antenna adapted to operate at radio frequencies.

In certain embodiments of the present invention, the structure is folded or rolled to provide the first configuration.

In certain embodiments of the present invention, the structure includes features to facilitate folding.

In certain embodiments of the present invention, the implantable medical device includes a carrier for conductive structure, and the features to facilitate folding are modifications to the conductive structure, the carrier, or both.

In certain embodiments of the present invention, part of the medical device is not adapted to change configuration.

In certain embodiments of the present invention, the structure is adapted to assume the second configuration automatically after insertion.

In embodiments of the present invention, a structure for use with an implantable device is provided, the structure having a first configuration and a second configuration, the first configuration presenting a smaller insertion profile than the second configuration, wherein after the structure is implanted, it is adapted to be placed into the second configuration.

In embodiments of the present invention, an electrical structure for use with an implantable medical device is provided, the resonant structure having a first configuration and a second configuration, the first configuration presenting a smaller insertion profile than the second configuration, wherein after the device is implanted, it is adapted to be placed into the second, operative configuration.

In embodiments of the present invention, a method of implanting a medical device is provided, the method including at least the steps of making an incision to as to access an appropriately sized internal opening in a body, providing an implantable medical device with a structure which has a first configuration and a second configuration, the implantable medical device being in the first configuration so as to present a smaller insertion profile than the second configuration, inserting the device through the incision and into the opening, the implantable medical device assuming a second, operative configuration within the opening in the body.

1-12. (canceled)

13. An implantable component of a medical device comprising:
an implantable coil comprising a conductor disposed in a carrier including first and second fold lines, wherein the carrier has at least one structural variation along each of the fold lines such that a substantially straight edge is formed along each of the fold lines when the coil is biased into a folded configuration; and
an electronics module electrically connected to the coil and configured to inductively communicate, via the coil, with an external component of the device.

14. The implantable component of claim 13, wherein the carrier is thinner at one or more regions of the carrier along each of the fold lines than at regions of the carrier adjacent at least one of the fold lines.

15. The implantable component of claim 13, wherein the carrier is thinner at one or more regions of the carrier where one of the fold lines crosses the conductor than at regions of the carrier adjacent at least one of the fold lines.

16. The implantable component of claim 15, wherein the cross-sectional area of the conductor is smaller at one or more regions of the conductor where one of the fold lines crosses the conductor than at regions of the conductor adjacent one or more of the fold lines.

17. The implantable component of claim 13, wherein the carrier has a first durometer at one or more regions along each of the fold lines and a second durometer at regions of the carrier adjacent at least one of the fold lines, and wherein the second durometer is greater than the first durometer.

18. The implantable component of claim 13, wherein the conductor is more flexible at one or more regions of the conductor where one of the fold lines crosses the conductor than at regions of the conductor adjacent one or more of the fold lines.

19. The implantable component of claim 13, wherein the carrier includes a plurality of apertures disposed along each of the first and second fold lines.

16. The implantable component of claim 14, wherein the carrier includes at least one channel is thinner at one or more regions where one of the fold lines crosses the conductor than at regions of the carrier adjacent at least one of the fold lines.

20. A system comprising:
an implantable component of a medical device including:
an implantable coil comprising a conductor disposed in a carrier including first and second fold lines, wherein the carrier has at least one structural variation along each of the fold lines such that a substantially straight edge is formed along each of the fold lines when the coil is biased into a folded configuration; and
an electronics module electrically connected to the coil and configured to inductively communicate, via the coil, with an external component of the device; and
an insertion tool configured to maintain the coil the folded configuration when the coil is operatively engaged with the insertion tool, wherein the coil assumes a substantially planar unfolded configuration when the coil is removed from operative engagement with the insertion tool.

21. The system of claim 20, wherein the insertion tool is a stylet, the coil comprises first and second lumens at first and second edges of the coil, the first and second lumens are configured to align, when the coil is in the folded state, to receive the stylet, and the stylet is configured to maintain the coil in the folded configuration when disposed in the first and second lumens.

22. The system of claim 20, wherein the insertion tool comprises a body and first and second flanges extending over the body, and wherein the insertion tool is configured to receive the coil in the folded configuration between the body and the flanges such that the flanges maintain the coil in the folded configuration.

23. The system of claim 20, wherein the memory of at least one of the carrier and the conductor causes the coil to assume the unfolded configuration when the coil is removed from operative engagement with the insertion tool.

24. The system of claim 20, wherein the coil comprises an adhesive disposed between adjacent portions of the coil when the coil is in the folded configuration, wherein the adhesive is configured to retain the coil in the folded configuration.

25. The system of claim 20, wherein the carrier is thinner at one or more regions of the carrier where one of the fold lines crosses the conductor than at regions of the carrier adjacent at least one of the fold lines.

26. The system of claim 25, wherein the conductor is more flexible at one or more regions of the conductor where one of the fold lines crosses the conductor than at regions of the conductor adjacent one or more of the fold lines.

27. A method of implanting a component of a medical device comprising an implantable coil including a conductor disposed in a carrier including first and second fold lines and an electronics module electrically connected to the coil and configured to inductively communicate, via the coil, with an external component of the device, the method comprising:
folding the coil, along each of the fold lines, into a folded configuration, wherein the carrier has at least one structural variation along each of the fold lines such that a substantially straight edge is formed along each of the fold lines when the coil is folded;
inserting the coil into an incision in a user's body along an axis substantially parallel to each of the fold lines; and
allowing the coil to assume an unfolded configuration in the user's body, wherein the width of the coil substantially perpendicular to the fold lines in the unfolded configuration is greater than the width of the coil substantially perpendicular to the fold lines in the folded configuration.

28. The method of claim 27, wherein the width of the coil substantially perpendicular to the fold lines in the unfolded configuration is greater than the width of the incision.

29. The method of claim 27, further comprising:
maintaining the coil in the folded configuration, using an insertion tool, prior to said inserting the coil into the incision, wherein said allowing the coil to assume an unfolded configuration comprises removing the insertion tool from the coil.

30. The method of claim 27, wherein the carrier is thinner at one or more regions of the carrier where one of the fold lines crosses the conductor than at regions of the carrier adjacent at least one of the fold lines.

31. The method of claim 30, wherein the conductor is more flexible at one or more regions of the conductor where one of the fold lines crosses the conductor than at regions of the conductor adjacent one or more of the fold lines.

32. The method of claim 31, wherein the conductor has a first cross-sectional shape at the one or more regions of the conductor where one of the fold lines crosses the conductor and has a second cross-sectional shape different than the first cross-sectional shape at the regions of the conductor adjacent one or more of the fold lines.
